(19) United States
(12) Patent Application Publication
(10) Pub. No.: US 2023/0255639 A1
(43) Pub. Date: Aug. 17, 2023

(54) APPLICATION SYSTEM FOR MEDICAL CLIPS

(71) Applicant: Aesculap AG, Tuttlingen (DE)

(72) Inventors: Thomas Pleil, Bad Duerrheim (DE); Thomas Scholten, Tuttlingen (DE); Michael Willmann, Villingen-Schwenningen (DE); Alexander Happle, Huefingen (DE); Jens Beger, Tuttlingen (DE)

(21) Appl. No.: 18/130,994

(22) Filed: Apr. 5, 2023

Related U.S. Application Data
(63) Continuation of application No. PCT/EP2021/077858, filed on Oct. 8, 2021.

(30) Foreign Application Priority Data
Oct. 9, 2020 (DE) 10 2020 126 581.6

(51) Int. Cl. A61B 17/128; A61B 17/122

(52) U.S. Cl. CPC A61B 17/128; A61B 17/1227

(57) ABSTRACT

An application system for medical clips includes at least one first application instrument for applying a first type of a medical clip and at least one first medical clip of the first type. The first application instrument includes two first tool elements that are movable relative to one another. The at least one first medical clip of the first type has a first proximal end region and two first connecting portions that distally adjoin the first proximal end region and connect the first proximal end region to two clamping arms of the at least one first clip of the first type that are movable relative to one another. The two first tool elements include between them a first end region receptacle that is open in the distal direction for accommodating the first proximal end region in a positive-locking or substantially positive-locking manner.

### CROSS-REFERENCE TO RELATED APPLICATIONS

This application is a continuation of International Application No. PCT/EP2021/077858 filed on Oct. 8, 2021 and claims priority to German Application No. 10 2020 126 581.6 filed on Oct. 9, 2020. The contents of International Application No. PCT/EP2021/077858 and German Application No. 10 2020 126 581.6 are incorporated by reference herein in their entireties and for all purposes.

### FIELD

The present invention relates to application systems for medical clips generally, and more specifically to an application system for medical clips, comprising at least one first application instrument for applying a first type of a medical clip and at least one first medical clip of the first type, wherein the first application instrument comprises two first tool elements that are movable relative to one another, wherein the at least one first medical clip of the first type has a first proximal end region and two first connecting portions, which distally adjoin the first proximal end region defining a first volume region and connect the first proximal end region to two clamping arms of the at least one first clip of the first type that are movable relative to one another, wherein the two first tool elements comprise between them a first end region receptacle that is open in the distal direction for accommodating the first proximal end region in a positive-locking or substantially positive-locking manner and each comprise a first connecting portion receptacle for at least partially accommodating a respective first accommodation portion of the first connecting portions.

### BACKGROUND

Application systems of the kind described at the outset are used, in particular, for the neurosurgical treatment of cranial aneurysms. They are used for the application of aneurysm clips, which are made of titanium or alloy available under the trademark PHYNOX™, for example.

One particular problem with such application systems is that they can fundamentally be used to apply different clip types. However, this is particularly problematic if the clip types differ, for example in size, shape and/or closing force, and the use of the known first application instrument can lead to damage to certain clip types.

### SUMMARY

In a first aspect of the invention, an application system for medical clips, comprising at least one first application instrument for applying a first type of a medical clip and at least one first medical clip of the first type, wherein the first application instrument comprises two first tool elements that are movable relative to one another, wherein the at least one first medical clip of the first type has a first proximal end region and two first connecting portions, which distally adjoin the first proximal end region defining a first volume region and connect the first proximal end region to two clamping arms of the at least one first clip of the first type that are movable relative to one another, wherein the two first tool elements comprise between them a first end region receptacle that is open in the distal direction for accommodating the first proximal end region in a positive-locking or substantially positive-locking manner and each comprise a first connecting portion receptacle for at least partially accommodating a respective first accommodation portion of the first connecting portions, wherein at least one first distinguishing element is arranged or formed on at least one of the two first tool elements in such a way that it engages into a second volume region, which is defined by a second proximal end region of a second medical clip of a second type, wherein two second connecting portions, each comprising a second accommodation portion, extend away from the second proximal end region in the distal direction, wherein the first volume region and the second volume region only partially overlap under the constraint that the second accommodation portions are each accommodated in a first respective connecting portion receptacle of the two first tool elements, wherein the application system comprises at least one second application instrument for applying a second type of a medical clip and at least one second medical clip of the second type, which comprises two second clamping arms that are movable relative to one another and that distally adjoin the two second connecting portions, wherein the at least one second application instrument comprises two second tool elements that are movable relative to one another, wherein the two second tool elements comprise between them an end region receptacle open in the distal direction for accommodating the second proximal end region in a positive-locking or substantially positive-locking manner and each comprise a second connecting portion receptacle for each at least partially accommodating a respective one of the second accommodation portions, wherein at least one second distinguishing element is arranged or formed on at least one of the two second tool elements in such a way that it engages into the first volume region under the constraint that the first accommodation portions are each accommodated in a respective second connecting portion receptacle of the two second tool elements.

### BRIEF DESCRIPTION OF THE DRAWING FIGURES

The foregoing summary and the following description may be better understood in conjunction with the drawing figures, of which:

FIG. 1 shows a schematic total view of a first embodiment of a first application instrument with a first medical clip of the first type;

FIG. 2 shows an enlarged perspective partial view of a distal end region of the first application instrument from FIG. 1 with an opened first end region receptacle for accommodating the first medical clip of the first type;

FIG. 3 shows a view of the arrangement from FIG. 2 without a clip from the front;

FIG. 4 shows a schematic view of the first application instrument from the front with a first medical clip of the first type corresponding to the first end region receptacle (left) and a second medical clip of the second type not corresponding to the first end region receptacle (right);

FIG. 5 shows an enlarged partial view of the distal end region of the first application instrument with a first medical clip of the first type accommodated therein;

FIG. 6: shows a view of the first application instrument from FIG. 4 from the front with a first medical clip of the first type accommodated therein;

FIG. 7 shows a partial view of the arrangement from FIG. 6 in the direction of the arrow A;

FIG. 8 shows a view similar to FIG. 7, but with tool elements moved somewhat toward one another;

FIG. 9 shows a schematic perspective view of a distal end region of the first application instrument when attempting to accommodate a second medical clip of the second type in the first end region receptacle;

FIG. 10 shows a view of the arrangement from FIG. 9 corresponding to FIG. 6;

FIG. 11 shows a schematic view similar to FIG. 4, but of a second application instrument with a first medical clip of the first type (left) and a second medical clip of the second type (right);

FIG. 12 shows an enlarged partial view of a distal end region of a further embodiment of a first application instrument with a further embodiment of a first medical clip of the first type;

FIG. 13 shows an enlarged perspective partial view of the distal end region of the application instrument from FIG. 12 with an opened first end region receptacle for accommodating the first medical clip of the first type from FIG. 12;

FIG. 14 shows a view of the first application instrument from FIGS. 12 and 13 from the front with a first medical clip of the first type with a lateral bead accommodated therein;

FIG. 15 shows a side view of the first medical clip of the first type from FIGS. 12 to 14;

FIG. 16 shows a view of the arrangement from FIG. 15 in the direction of the arrow A;

FIG. 17 shows a view of the arrangement from FIG. 15 in the direction of the arrow B;

FIG. 18 shows an enlarged partial view of the first tool element of the first application instrument from FIGS. 12 to 14;

FIG. 19 shows a view in the direction of the arrow C from FIG. 18;

FIG. 20 shows a schematic enlarged view of first tool elements of a further embodiment of a first application instrument; and

FIG. 21 shows a schematic view of the first application instrument from FIG. 20 from the front with a first medical clip of the first type corresponding to the first end region receptacle (left) and a second medical clip of the second type not corresponding to the first end region receptacle (right).

### DETAILED DESCRIPTION

Although the invention is illustrated and described herein with reference to specific embodiments, the invention is not intended to be limited to the details shown. Rather, various modifications may be made in the details within the scope and range of equivalents of the claims and without departing from the invention.

The present invention relates to an application system for medical clips, comprising at least one first application instrument for applying a first type of a medical clip and at least one first medical clip of the first type, wherein the first application instrument comprises two first tool elements that are movable relative to one another, wherein the at least one first medical clip of the first type has a first proximal end region and two first connecting portions, which distally adjoin the first proximal end region defining a first volume region and connect the first proximal end region to two clamping arms of the at least one first clip of the first type that are movable relative to one another, wherein the two first tool elements comprise between them a first end region receptacle that is open in the distal direction for accommodating the first proximal end region in a positive-locking or substantially positive-locking manner and each comprise a first connecting portion receptacle for at least partially accommodating a respective first accommodation portion of the first connecting portions, wherein at least one first distinguishing element is arranged or formed on at least one of the two first tool elements in such a way that it engages into a second volume region, which is defined by a second proximal end region of a second medical clip of a second type, wherein two second connecting portions, each comprising a second accommodation portion, extend away from the second proximal end region in the distal direction, wherein the first volume region and the second volume region only partially overlap under the constraint that the second accommodation portions are each accommodated in a first respective connecting portion receptacle of the two first tool elements, wherein the application system comprises at least one second application instrument for applying a second type of a medical clip and at least one second medical clip of the second type, which comprises two second clamping arms that are movable relative to one another and that distally adjoin the two second connecting portions, wherein the at least one second application instrument comprises two second tool elements that are movable relative to one another, wherein the two second tool elements comprise between them an end region receptacle open in the distal direction for accommodating the second proximal end region in a positive-locking or substantially positive-locking manner and each comprise a second connecting portion receptacle for each at least partially accommodating a respective one of the second accommodation portions, wherein at least one second distinguishing element is arranged or formed on at least one of the two second tool elements in such a way that it engages into the first volume region under the constraint that the first accommodation portions are each accommodated in a respective second connecting portion receptacle of the two second tool elements.

The proposed further development of an application system for medical clips of the kind described at the outset makes it possible, in particular, to prevent the second proximal end region of the second medical clip of the second type from being able to be accommodated under the specified constraint, that is, that the second accommodation portions are each accommodated in a respective one of the two first connecting portion receptacles of the two first tool elements. Since the second proximal end region of the second clip of the second type cannot be accommodated in the first end region receptacle under the constraint, it also cannot be applied, especially unintentionally. The proposed further development enables, in particular, a reliable mechanical and thus automatic differentiation of different clip types with the first application instrument. The at least one first distinguishing element thereby prevents, in particular, that the second proximal end region of the second medical clip of the second type can be accommodated in the first end region receptacle. In contrast to, for example, coding of application instruments and clips with colors, in particular, no confusion can occur, since the at least one first distinguishing element prevents a medical clip that is not of the first type from being able to be accommodated by the first application instrument. In particular, the at least one first distinguishing element may be arranged or formed in such a way that when moving the first tool elements toward one another, the clip, which is not of the first type, is pushed out or jumps out of the first end region receptacle. This allows a surgeon to immediately recognize that an attempt has been made to apply the wrong clip type. The application system configured as proposed makes it possible, in particular, to use technical design features of different medical clips, for example different generations of clips, to enable a defined association with corresponding application instruments, thereby minimizing a risk of confusion for the application of different medical clips, i.e. in particular of different types. In particular, such design differences can be realized in the proximal end region of the different clip types. The at least one first distinguishing element may, in particular, make the use of the first application instrument with medical clips that are not of the first type more difficult or even impossible.

It is favorable that the application system comprises at least one second application instrument for applying a second type of a medical clip and at least one second medical clip of the second type, which comprises two second clamping arms that are movable relative to one another and that distally adjoin the two second connecting portions, that the at least one second application instrument comprises two second tool elements that are movable relative to one another, that the two second tool elements comprise between them a second end region receptacle open in the distal direction for accommodating the second proximal end region in a positive-locking or substantially positive-locking manner and each comprise a second connecting portion receptacle for at least partially accommodating a respective one of the second accommodation portions, that at least one second distinguishing element is arranged or formed on at least one of the two second tool elements in such a way that it engages into the first volume region under the constraint that the first accommodation portions are each accommodated in a respective second connecting portion receptacle of the two second tool elements. The at least one second application instrument is therefore suitable, in particular, in an analogous manner as the first application instrument exclusively for accommodating and applying a second medical clip of the second type. Medical clips of the first type cannot be accommodated or can be accommodated only to a very limited extent with their first proximal end region in the second end region receptacle. Preferably, the at least one second distinguishing element is arranged in such a way that the at least one first medical clip of the first type jumps out of the second end region receptacle of the second application instrument when it is gripped with the second application instrument. With such a system, at least two different clip types can be reliably distinguished and applied in a defined and reliable manner with the at least two application instruments corresponding to said clip types. Thus, in particular, in an operating room where there are often only limited visual conditions, in addition to optical distinguishing features that may optionally be provided or result from a different design of the clip types, a reliable mechanical differentiation between the clip types can be realized with the two application instruments. The distinguishing elements may be configured, in particular, in the form of projections, set-back portions, or specially shaped abutment surfaces, which mechanically interact with certain shapes of the clip types, for example shapes of end regions thereof, and either enable the complete accommodation of the medical clip of a particular type by the associated application instrument in order to be able to apply the clip, or not.

It is advantageous if the at least one second application instrument comprises a second actuating device for moving, in particular for pivoting, the two second tool elements relative to one another from a second accommodation position, in which a second medical clip of the second type corresponding to the second application instrument can be accommodated with its second end region in the second end region receptacle, into a second application position, in which the two second tool elements are moved toward one another and the second clamping arms are moved away from one another for applying the second clip of the second type. An application instrument configured in that way enables, in particular, simple and reliable handling of second medical clips of the second type in order to be able to reliably apply them in the desired manner.

In order to be able to securely handle second medical clips of the second type, in particular in such a way that they cannot unintentionally fall off from the second application instrument, it is advantageous if the second tool elements of the second application instrument partially engage around the second proximal end region in the second accommodation position and in the second application position.

Favorably, the two second tool elements of the at least one second application instrument are movable relative to one another, in particular are pivotable relative to one another about a common second pivot axis. In this way, any second application instruments and, in particular, second application instruments that can be easily and reliably handled, can be formed. In particular, second tool elements that are pivotable relative to one another can be moved sensitively relative to one another in a defined manner.

In accordance with a further preferred embodiment of the invention, provision may be made that the at least one first application instrument comprises a first actuating device for moving, in particular for pivoting, the two first tool elements relative to one another from a first accommodation position, in which a first medical clip of the first type corresponding to the first application instrument can be accommodated with its first end region in the first end region receptacle, into a first application position, in which the two first tool elements are moved toward one another and the first clamping arms are moved away from one another for applying the first clip of the first type. With a first application instrument configured in this way, medical clips of the first type can be handled easily and securely and can be applied in a defined and desired manner.

In order to prevent an unintentional release of medical clips of the first type from the first application instrument as far as possible, it is advantageous if the first tool elements of the first application instrument partially engage around the first proximal end region in the first accommodation position and in the first application position.

It is advantageous if the two first tool elements of the at least one first application instrument are movable relative to one another. In particular, it is advantageous if they are pivotable relative to one another about a common first pivot axis. First tool elements configured in that way make it possible, in particular, to accommodate a proximal end region of a first medical clip of the first type and to apply it through a relative movement of the two first tool elements to one another, for example a pivot movement.

It is favorable if the first accommodation portions directly or substantially directly adjoin the first proximal end region and/or if the second accommodation portions directly or substantially directly adjoin the second proximal end region. Configuring medical clips in this way makes it possible, in particular, to accommodate end regions of the medical clips in the respective end region receptacle and to engage on the clips at the respective accommodation portions with regions of the tool elements of the respective application instruments, said regions pointing in the distal direction, the application instruments each having a respective connecting portion receptacle, in order to open and close the clips accommodated by the respective application instrument by moving the tool elements relative to one another.

The first proximal end region favorably comprises a first biasing element and/or the second proximal end region comprises a second biasing element. The proximal end region thus serves, in particular, not only for distinguishing medical clips of different types, but, in particular, also for biasing the clamping arms against one another. In particular, the clamping arms of the medical clips can be moved away from one another against the action of the biasing elements. The opening force required for this can be introduced, for example, by way of the connecting portion receptacles of the tool elements of the application instruments to the accommodation portions of the medical clips.

It is advantageous if the first clamping arms abut against one another in a first basic position of the at least one first clip of the first type and are movable away from one another against the action of the first biasing element from the basic position into a first opening position and/or if the second clamping arms abut against one another in a second basic position of the at least one second clip of the second type and are movable away from one another against the action of the second biasing element from the second basic position into a second opening position. As already explained, the clips can thus be opened and closed again in a defined manner using an application instrument configured correspond thereto. In particular, the biasing element can also predetermine a secure positioning of the respective clip between the associated tool elements. In particular, a clip can thus always be held clamped to the application instrument independently of whether it is in the basic position or an opening position.

In accordance with a further preferred embodiment of the invention, provision may be made that the first connecting portions intersect in a first connection region of the at least one first clip of the first type and/or that the second connecting portions intersect in a second connection region of the at least one second clip of the second type. A design of that kind makes it possible, in particular, for the clamping arms of the respective clips to be able to be moved away from one another by moving the accommodation portions toward each other when these are arranged or formed on the proximal side of the connection region. To apply the clips, the tool elements of the respective application instrument can then be moved toward one another. The clip is thereby opened. The tool elements are moved away from one another again for application. In particular, the biasing element then leads to the clamping arms being moved back toward one another, in particular so far that they, as the case may be, abut against one another or are able to clamp between them a hollow organ, for example a blood vessel or a sacculation thereof.

Preferably, the first connection region is arranged or formed on the distal side of the first accommodation portions and/or the second connection region is arranged or formed on the distal side of the second accommodation portions. As already explained, the clamping arms can thus be moved away from one another by the respective accommodation portions being moved toward one another, for example by moving the cooperating tool elements of the respective application instrument toward one another.

The first connection region and/or the second connection region are favorably configured in the form of a box lock. Clips with box locks have the advantage, in particular, that the connecting portions are guided on one another. In this way, unwanted torsions in the region of the clamping arms can be avoided or largely avoided. This is particularly advantageous, since so-called “scissoring” can be avoided. In other words, mutual shearing of the clamping arms when closing the clip can be prevented, which can injure tissue clamped between the clamping arms.

The clips can be formed in a simple manner if the first biasing element is configured in the form of a first coil spring with at least one complete winding and/or if the second biasing element is configured in the form of a second coil spring with at least one complete winding. By selecting the material from which the coil spring is formed, as well as its size, the clamping forces of the clips can be predetermined in the desired manner. Furthermore, the shape of the respective coil spring can be used to distinguish clips of different types from one another with the application instruments of the application system.

The clips can be formed in a simple and cost-effective manner if the first biasing element and/or the second biasing element are formed by a cold forming process. For example, the biasing elements can be configured in the form of coil springs by winding.

In accordance with a further preferred embodiment of the invention, provision may be made that the at least one first medical clip of the first type defines a first clip plane, that the first coil spring defines a first coil spring longitudinal axis, that the first coil spring longitudinal axis extends perpendicularly to a first coil spring plane, and that the first coil spring plane is inclined relative to the first clip plane by a first angle of inclination, and/or that the at least one second medical clip of the second type defines a second clip plane, that the second coil spring defines a second coil spring longitudinal axis, that the second coil spring longitudinal axis extends perpendicularly to a second coil spring plane, and that the second coil spring plane is inclined relative to the second clip plane by a second angle of inclination. The medical clips of the first type and of the second type can thus be distinguished, in particular, by different angles of inclination of the respective coil spring plane relative to the respective clip plane. The clip plane can be defined, in particular, by a plane spanned by the accommodation portions of the clips. In particular, the clips may be configured in such a way that the accommodation portions move in the clip plane when opening and closing the clips. For example, the clamping arms may extend in the clip plane or, for example, in certain embodiments, may also be curved out of the clip plane.

It is favorable if the first coil spring is wound clockwise and if the first angle of inclination has a positive value and/or if the second coil spring is wound counterclockwise and if the second angle of inclination has a negative value. This further development makes it possible, in particular, to distinguish clips of different types in a simple manner. This can be achieved, in particular, due to the angles of inclination. These can be realized in a simple manner through different winding directions, i.e. once clockwise and once counter-clockwise with respect to the coil spring longitudinal axis. Due to the different angles of inclination, the coil spring, which ultimately forms at least part of the end region of the respective clip, defines a volume region. However, volume regions with different angles of inclination do not overlap, such that the clips of different types can be mechanically distinguished in a simple and secure manner due to corresponding distinguishing elements on the respective application instruments.

A secure differentiation of different clip types can be ensured, in particular, by the first angle of inclination having a value in a range between 0° and about 40°, in particular a value in a range between 0° and about 20°, and/or by the second angle of inclination having a value in a range between 0° and about

−40°, in particular a value in a range between 0° and about −20°. Angles of inclination with positive and negative values thus define inclinations of the coil spring planes relative to the respective clip plane in different directions.

It is advantageous if the at least one first application instrument is configured corresponding to a first medical clip of the first type with a first coil spring wound clockwise.

In this way, an unambiguous association between medical clips of the first type and the associated first application instrument can be achieved.

The at least one second application instrument is preferably configured corresponding to a second medical clip of the second type with a second coil spring wound counter-clockwise. With a corresponding second application instrument, second medical clips of the second type can thus be handled and, in particular, applied in a simple and reliable manner.

In accordance with a further preferred embodiment of the invention, provision may be made that the at least one first distinguishing element is configured in the form of a first coding element that cooperates with any medical clip and that exclusively the first medical clip of the first type corresponding to the first coding element can be accommodated in the first end region receptacle of the first application instrument in a positive-locking or substantially positive-locking manner. In particular, the proximal end region of the first medical clip of the first type can be accommodated in the first end region receptacle, since the first coding element does not engage into the first volume region defined by the first end region. By contrast, it is not possible to insert a second medical clip of the second type into the first end region receptacle under the constraint that the accommodation portions are accommodated in the connecting portion receptacles of the first application instrument. In this case, the first coding element protrudes into the second volume region, which is defined by the second end region of the second medical clip of the second type. As a result, the second end region cannot be accommodated in the first end region receptacle.

In order to enable a defined positioning of the first end region in the first end region receptacle, it is favorable if the at least one first coding element is configured in the form of a first coding projection, which laterally delimits the first end region receptacle at least in sections. In particular, tilting of the clip in the first end region receptacle can thus be prevented.

It is advantageous if the first application instrument comprises two first coding elements that cooperate with any medical clip and if the first coding elements form lateral abutment surfaces for sides pointing away from one another exclusively for the first end region accommodated in the first end region receptacle. The lateral abutment surfaces of the first coding elements can thus, in particular, prevent the first medical clip of the first type from being rotated about an axis lying in the first clip plane when it is accommodated in the first end region receptacle. This enables a reliable application of first medical clips of the first type with the first application instrument.

It may further be advantageous if the at least one second distinguishing element is configured in the form of a second coding element that cooperates with any medical clip and if exclusively the second medical clip of the second type corresponding to the second coding element can be accommodated in the second end region receptacle of the second application instrument in a positive-locking or substantially positive-locking manner. The second coding element thus makes it possible, in particular, to distinguish between second medical clips of the second type and any other clips. Thus, in particular, a mechanical association of the second application instrument and second medical clips of the second type can be achieved in a simple manner.

The at least one second coding element is preferably configured in the form of a second coding projection, which laterally delimits the second end region receptacle at least in sections. For example, a second end region of a second clip of the second type can be supported on the second coding projection when the second end region is accommodated in the second end region receptacle of the second application instrument.

It is favorable if the second application instrument comprises two second coding elements that cooperate with any medical clip and if the second coding elements form lateral abutment surfaces for sides pointing away from one another exclusively for the second end region accommodated in the second end region receptacle. In this way, in particular, it can be achieved, in particular, that the clip cannot rotate about an axis that lies in the second clip plane when the second end region is accommodated in the second end region receptacle of the second application instrument.

It is advantageous if a respective one of the two first coding elements is arranged or formed on each first tool element of the first application instrument and/or if a respective one of the two second coding elements is arranged or formed on each second tool element of the second application instrument. Thus, the clips corresponding to the respective tool elements can already be guided in a defined manner when their proximal end regions are accommodated into the respective end region receptacle. If, for example, an attempt is made to accommodate a clip, which does not correspond to the respective application instrument, with its proximal end region in the end region receptacle of the respective application instrument, the coding elements interact with the respective end region of the non-matching clip and lead to the clip being rotated and falling out of the respective end region receptacle or not being able to be accommodated therein.

It is advantageous if the at least one first coding element is formed on the one of the two first tool elements projecting in the distal direction beyond a proximal end of the other first tool element delimiting the first end region receptacle and/or if the at least one second coding element is formed on the one of the two second tool elements projecting in the distal direction beyond a proximal end of the other second tool element delimiting the second end region receptacle. Such a design makes it possible, in particular, to mechanically detect geometric differences relative to the respective clip plane. For example, depending on whether the end region has a coil spring inclined in one direction or the other, the respective coding elements allow or prevent the accommodation of an end region of a medical clip in the end region receptacle.

In accordance with a further preferred embodiment of the invention, provision may be made that the two first tool elements are convertible into one another in the region of the first end region receptacle by rotation by 180° relative to a first longitudinal axis of the first end region receptacle and/or that the two second tool elements are convertible into one another in the region of the second end region receptacle by rotation by 180° relative to a second longitudinal axis of the second end region receptacle. This design simplifies, in particular, a construction of the respective application instruments. For example, it is possible to use two identical tool elements to form the respective application instruments.

For treating aneurysms, it is advantageous, in particular, if the at least one first medical clip of the first type and/or if the at least one second medical clip of the second type are configured in the form of aneurysm clips.

An embodiment of an application system 10 is schematically depicted in the Figures. It serves for the application of medical clips.

The application system 10 comprises a first application instrument 12 for applying a first medical clip 14 of a first type. As explained in more detail in the following, the first application instrument 12 is configured to distinguish a first clip 14 from a second medical clip 16 of a second type.

The first application instrument 12 comprises two first tool elements 18 that are movable relative to one another.

The first clip 14 of the first type has a first proximal end region 20 and two first connecting portions 22. The two first connecting portions 22 distally adjoin the first end region 20.

The first end region 20 defines a first volume region 24. The two first connecting portions 22 connect the first end region 20 to a respective first clamping arm 26.

The two first tool elements 18 define between them a first end region receptacle 28 open in the distal direction. The first end region 20 can be accommodated therein in a positive-locking or substantially positive-locking manner.

A first connecting portion receptacle 30 is formed on each of the two tool elements 18. It serves to accommodate at least part of a first accommodation portion 32 of the first connecting portions 22.

Furthermore, a respective first distinguishing element 34 is arranged or formed on the first tool elements 18. The first distinguishing elements 34 are arranged or formed in such a way that they engage or protrude into a second volume region 36, which is defined by a second proximal end region 38 of the second clip 16 of the second type.

Two second connecting portions 40 extend in the distal direction away from the second proximal end portion 38 in the distal direction. The second connecting portions 40 each comprise a second accommodation portion 42.

The first volume region 24 and the second volume region 36 overlap only partially under the constraint that the second accommodation portions 42 are each accommodated in one of the first connecting portion receptacles 30 of the two first tool elements 18. This prevents the second clip 16 of the second type from being able to be accommodated with its second proximal end region 38 in the first end region receptacle 28.

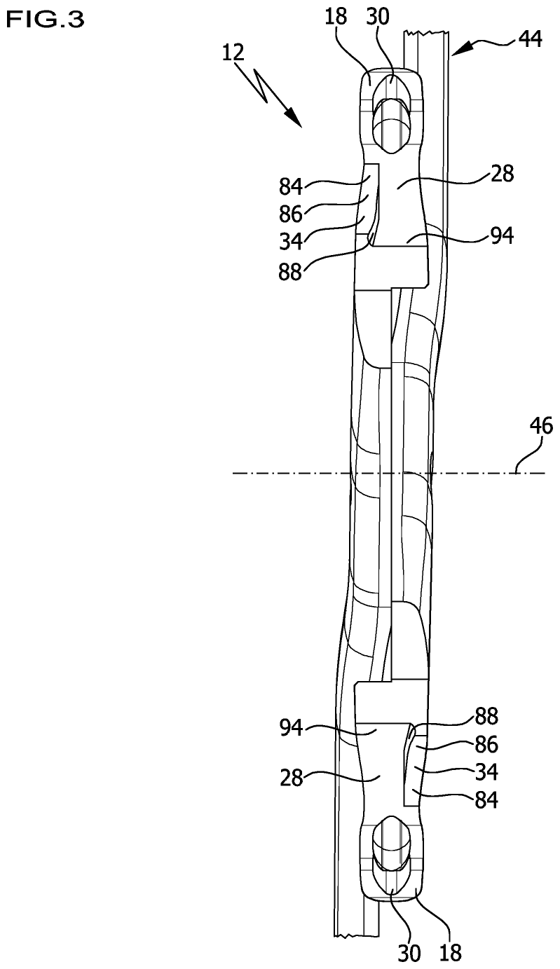

The first application instrument 12 comprises a first actuating device 44 for moving, namely for pivoting, the two first tool elements 18 relative to one another from a first accommodation position, as it is shown schematically in FIG. 3 and in which a first medical clip 14 of the first type corresponding to the first application instrument 12 can be accommodated with its first end region 20 in the first end region receptacle 28, into a first application position, which is schematically shown in FIG. 8 and in which the two first tool elements 18 are moved slightly toward one another and the first clamping arms 26 are moved slightly away from one another, so that the first clip 14 of the first type can be applied.

The first tool elements 18 of the first application instrument 12 partially engage around the first proximal end region 20 in the first accommodation position and in the first application position.

In the case of the first application instrument 12 depicted in the Figures, the two first tool elements 18 are pivotable relative to one another about a common first pivot axis 46.

In the case of the first clips 14 of the first type schematically depicted in the Figures, the first accommodation portions 32 directly or substantially directly adjoin the first proximal end region 20. In a corresponding manner, the second accommodation portions 42 directly or substantially directly adjoin the second proximal end region 38.

The first proximal end region 20 comprises a first biasing element 48 in the form of a first coil spring 50 with about one and a half turns.

The second end region 38 comprises in a corresponding manner a second biasing element 52, which is configured in the form of a second coil spring 54, also with about one and a half turns.

In the described embodiment, the biasing elements 48 and 52 are formed by a cold forming process.

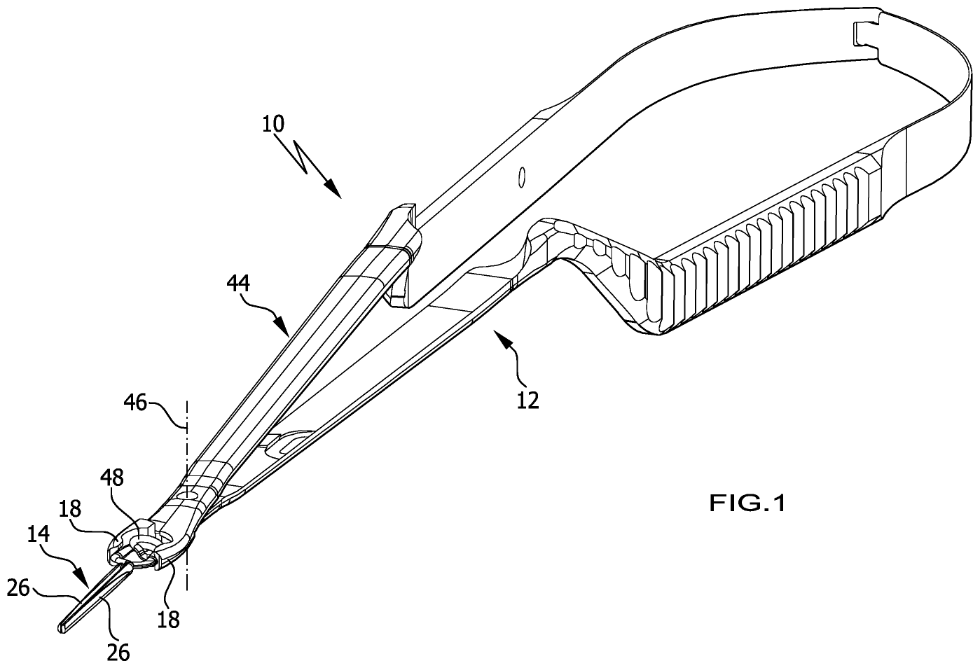

The first clamping arms 26 abut against one another in a first basic position of the first clip 14 of the first type, as schematically shown, in particular, in FIG. 1. The clamping arms 26 are movable away from one another against the action of the first biasing element 48 from the first basic position into any first opening position. Such a first opening position is schematically depicted, for example, in FIG. 8.

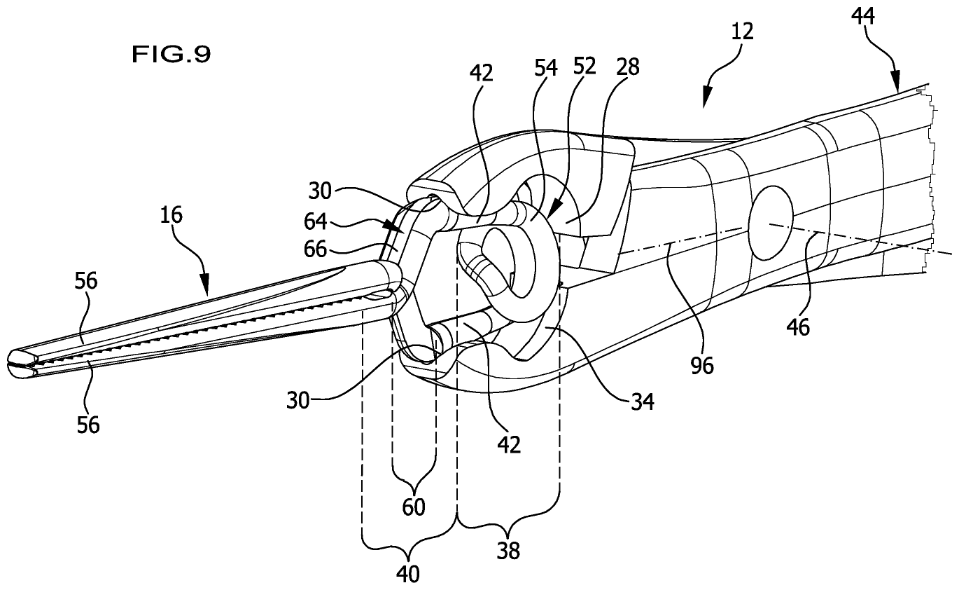

The second clip 16 of the second type has two second clamping arms 56, which abut against one another in a basic position, as schematically depicted in FIG. 9, and are movable away from one another against the action of the second biasing element 52 from the second basic position into any second opening position, i.e. analogous to a position of the first clip 14 as is schematically depicted in FIG. 8.

In the embodiments depicted in the Figures of the first clip 14 of the first type and of the second clip 16 of the second type, the first connecting portions 22 intersect in a first connection region 58. In an analogous manner, the second connecting portions 40 intersect in a second connection region of the second medical clip 16 of the second type.

The first connection region 58 is arranged or formed on the distal side of the first accommodation portions 32. In an analogous manner, the second connection region 60 is arranged or formed on the distal side of the second accommodation portions 42.

The connection regions 58 and 60 are configured in the form of box locks 62 and 64, respectively. Here, one of the connecting portions 22 or 40 has a connection region perforation 66, which is passed through by the respective other connecting portion 22 or 40.

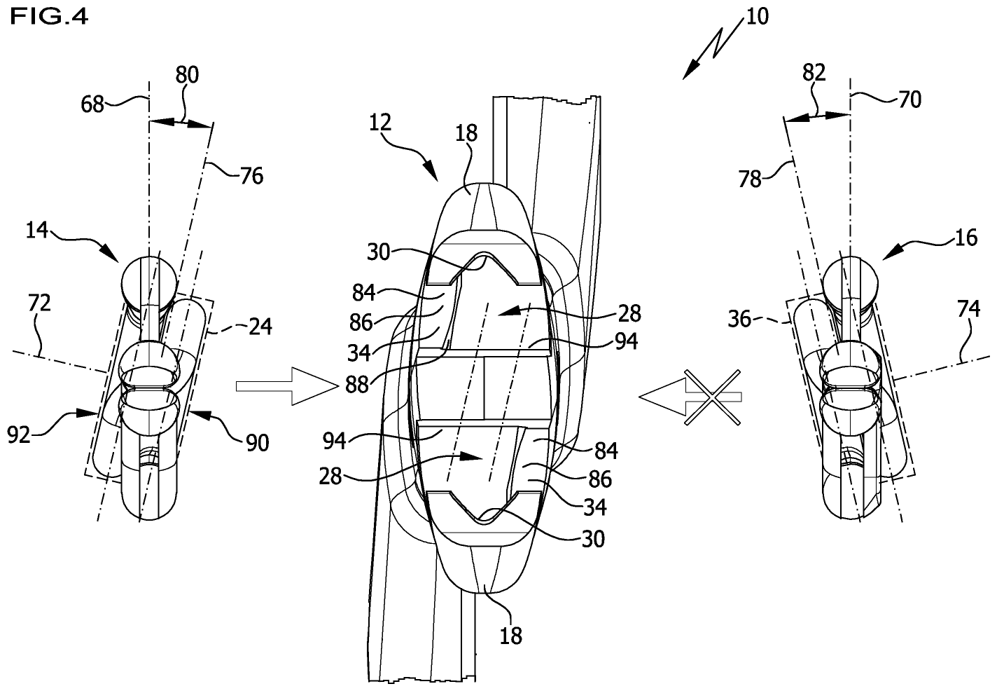

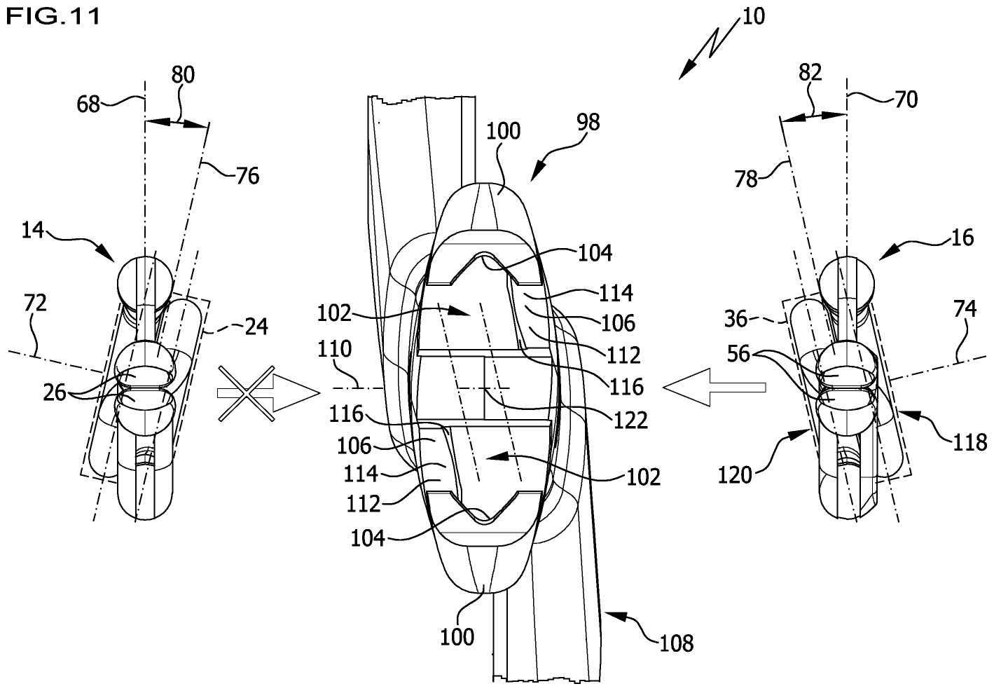

With reference to FIGS. 4 and 11, the first medical clip 14 of the first type defines a first clip plane 68. Said clip plane 68 is spanned by the two first accommodation portions 32.

In an analogous manner, the second medical clip 16 defines a second clip plane 70.

The first coil spring 50 defines a first coil spring longitudinal axis 72, about which the first coil spring 50 is wound clockwise.

The second coil spring 54 defines a second coil spring longitudinal axis 74, about which the second coil spring 54 is wound counter-clockwise.

The first coil spring longitudinal axis 72 extends perpendicularly to a first coil spring plane 76. Likewise, the second coil spring longitudinal axis 74 extends perpendicularly to a second coil spring plane 78.

The first coil spring plane 76 is inclined relative to the first clip plane 68 by a first angle of inclination 80. The second coil spring plane 78 is inclined relative to the second clip plane 70 by a second angle of inclination 82.

In the case of the first coil spring 50 wound clockwise, the first angle of inclination 80 has a positive value, which is in a range between 0° and about 40°. In the embodiment of the first clip 14 depicted in the Figures, the value of the angle of inclination 80 is in a range between 0° and about 20°.

In the case of the second coil spring 54 wound counter-clockwise, the second angle of inclination 82 has a negative value, which is in a range between 0° and about −40°. In the embodiment depicted in the Figures, the second angle of inclination 82 is in a range between 0° and −20°.

In the application system 10, the first application instrument 12 is configured corresponding to the first medical clip 14 of the first type with a first coil spring 50 wound clockwise.

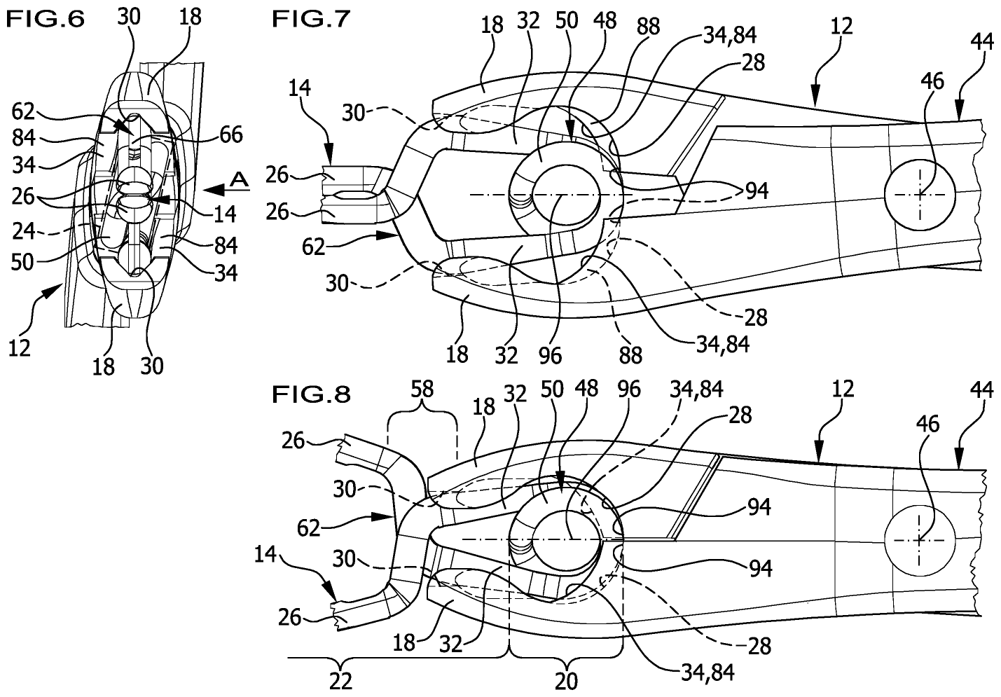

The first distinguishing elements 34 are configured in the form of first coding elements 84. They are arranged and configured in such a way that exclusively a first medical clip 14 of the first type corresponding to the first coding elements 84 can be accommodated in the first end region receptacle 28 of the first application instrument 12. This is shown schematically in FIG. 6. Furthermore, FIG. 4 shows on the left the first clip 14 of the first type, which is insertable into the first end region receptacle 28.

However, the first distinguishing elements 34 also cooperate with other clips, in particular also with the second medical clips 16 of the second type.

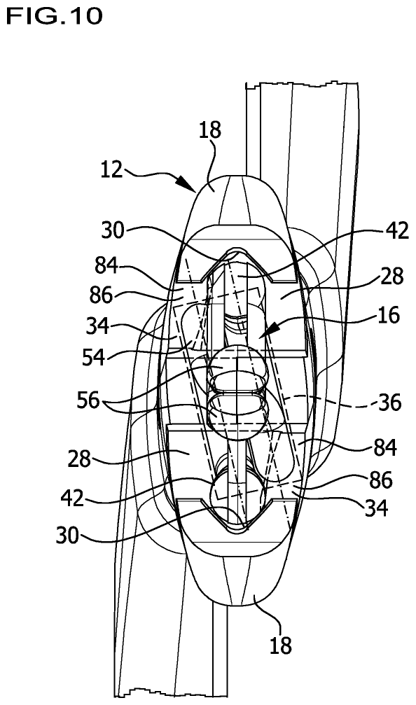

In cooperation with a second medical clip 16, the first coding elements 84 prevent the insertion of the second end region 38 into the first end region receptacle 28. In FIG. 4 on the right, the second medical clip 16 of the second type is shown, the second end region 38 of which does not correspond to the first end region receptacle 28. This only allows the accommodation of the first volume region 24, which is defined by the first end region 20, when the first accommodation portions 32 are accommodated in the first connecting portion receptacles 30 of the first tool elements 18. By contrast, the coding elements 84 collide with the second volume region 36, which is defined by the second end region 38 of the second medical clip 16 of the second type, when the second accommodation portions 42 engage into the first connecting portion receptacles 30. This is immediately recognizable, in particular, in FIGS. 4 and 10.

It should be noted that FIG. 10 shows a situation that is not really possible, since the second end region 38 and the coding elements 84 overlap. Here it can be clearly seen, in particular in FIG. 10, that the coding element 84 engages into the second volume region 36 under the described constraint that the second accommodation portions 42 are accommodated in the first connecting portion receptacles 30. As already explained, this collision is not really possible. Rather, this design of the first application instrument 12 in cooperation with the second medical clip 16 of the second type leads to said second medical clip 16 being pushed with its second end region 38 out of the first end region receptacle 28.

The first coding element 84 is configured in the form of a first coding projection 86, which laterally delimits the first end region receptacle 28 in sections.

The first application instrument 12 comprises two cooperating first coding elements 84, which form lateral abutment surfaces 88 for sides 90 and 92 of the first end region 20 accommodated in the first end region receptacle 28, said sides pointing away from one another.

A coding element 84 is arranged or formed on each of the two first tool elements 18 in the manner described.

Furthermore, the first coding element 84 on the one of the two first tool elements 18 projects in the distal direction beyond a proximal end 94 of the other first tool element 18 delimiting the first end region receptacle 28.

The two first tool elements 18 are configured identical in their form and are convertible into one another in the region of the first end region receptacle 28 by rotation by 180° relative to a first longitudinal axis 96 of the first end region receptacle 28.

The two clips 14 and 16 are each configured in the form of aneurysm clips.

The application system 10 schematically depicted in the Figures further comprises a second application instrument 98 for applying the second medical clip 16 of the second type.

The second application instrument 98 comprises two second tool elements 100 that are movable relative to one another. The two second tool elements 100 define between them a second end region receptacle 102 open in the distal direction, in which the second proximal end region 38 can be accommodated in a positive-locking or substantially positive-locking manner.

A respective second connecting portion receptacle 104 is formed on each of the second tool elements 100. These are oriented pointing toward one another and are intended to each accommodate one of the second accommodation portions 42 of the second medical clip 16 of the second type.

Second distinguishing elements 106 are arranged or formed on the two second tool elements 100 in such a way that they engage into the first volume region 24 under the constraint that the first accommodation portions 32 are each accommodated in a respective one of the second connecting portion receptacles 104. In other words, the second application instrument 98 is configured in such a way that, under the described constraint, it is only suitable to accommodate the second medical clip 16 of the second type with its second end region 38 in the second end region receptacle 102, but not the first end region 20 of the first medical clip 14 of the first type.

The application instruments 12 and 98 are therefore suitable for mechanically and, in particular, automatically distinguishing the clips 14 and 16 from one another.

The second application instrument 98 comprises a second actuating device 108 for moving, in the embodiment depicted in FIG. 11 for pivoting, the second tool elements 100 relative to another about a second pivot axis 110. Using the actuating device 108, the two second tool elements 100 can be pivoted relative to one another from a second accommodation position, as it is schematically depicted in FIG. 11 and in which a second medical clip 16 of the second type corresponding to the second application instrument 98 can be accommodated with its second end region 38 in the second end region receptacle 102, into a second application position, in which the two second tool elements 100 are moved toward one another and the second clamping arms 56 are moved away from one another, in order to be able to apply the second clip 16 of the second type.

The second tool elements 100 partially engage around the second proximal end region 38 in the second accommodation position and in the second application position.

As already explained above and as is directly recognizable from FIG. 11, the second application instrument 98 is configured corresponding to the second medical clip 16 of the second type, which has a second coil spring 54 wound counter-clockwise.

The second distinguishing element 106 is configured analogously to the first distinguishing element 34 in the form of a second coding element 112, which cooperates with any medical clips. However, the second coding element 112 in the region of the second end region receptacle 102 of the second application instrument 98 is arranged or formed in such a way that exclusively the second medical clip 16 of the second type corresponding to the second coding element 112 can be accommodated in the second end region receptacle 102 in a positive-locking or substantially positive-locking manner.

The second coding element 112 is also configured in the form of a second coding projection 114, which laterally delimits the second end region receptacle 102 in sections.

The second application instrument 98 schematically depicted in the Figures has two second coding elements 112. These define lateral abutment surfaces 116, which face toward one another, for sides 118 and 120, which face away from one another, of the second end region 38 accommodated in the second end region receptacle 102.

As explained above in conjunction with FIG. 7 and the first application instrument 12, the second coding elements 112 are also formed on the respective second tool element 100 projecting in the distal direction beyond a proximal end of the other second tool element 100 delimiting the second end region receptacle 102.

The two second tool elements 100 are also convertible into one another in the region of the second end region receptacle 102 by rotation by 180° relative to a second longitudinal axis 122, which extends in parallel to the second clip plane 70.

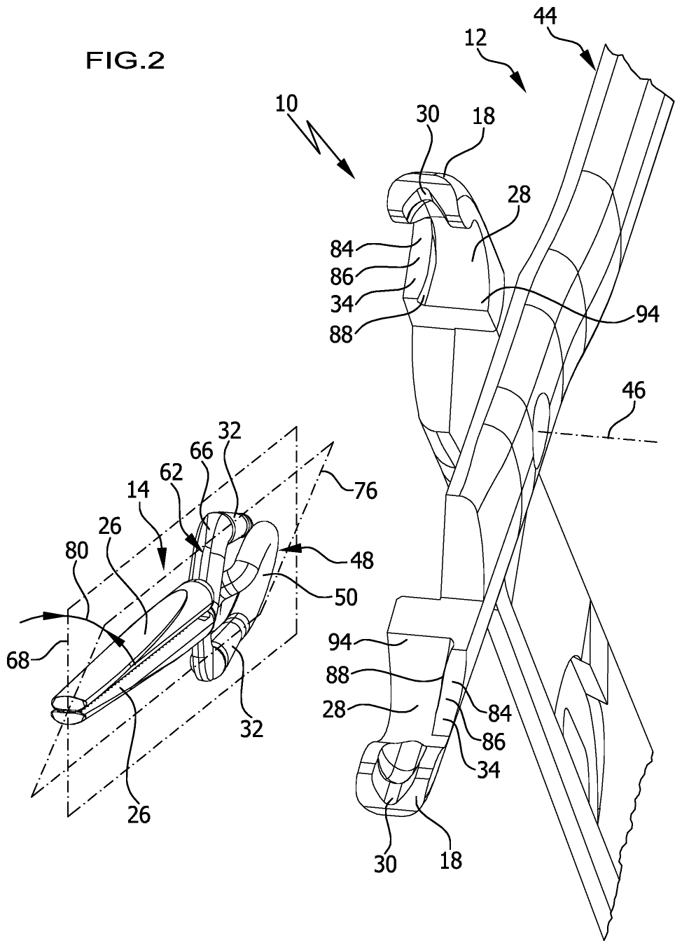

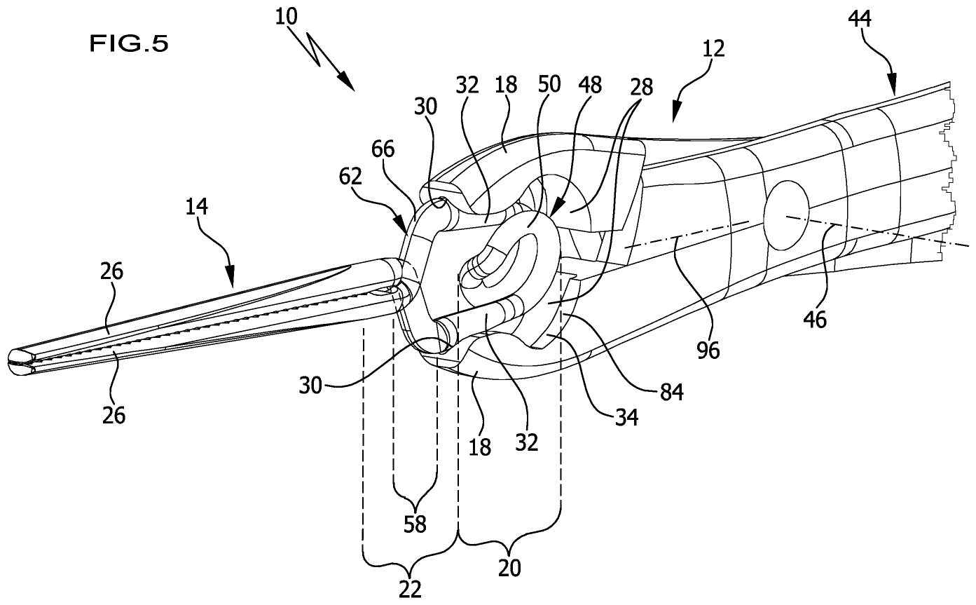

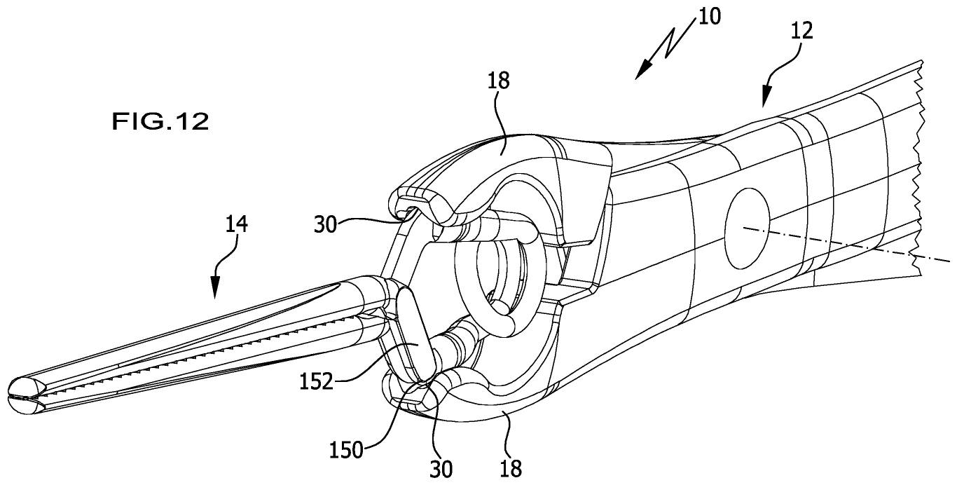

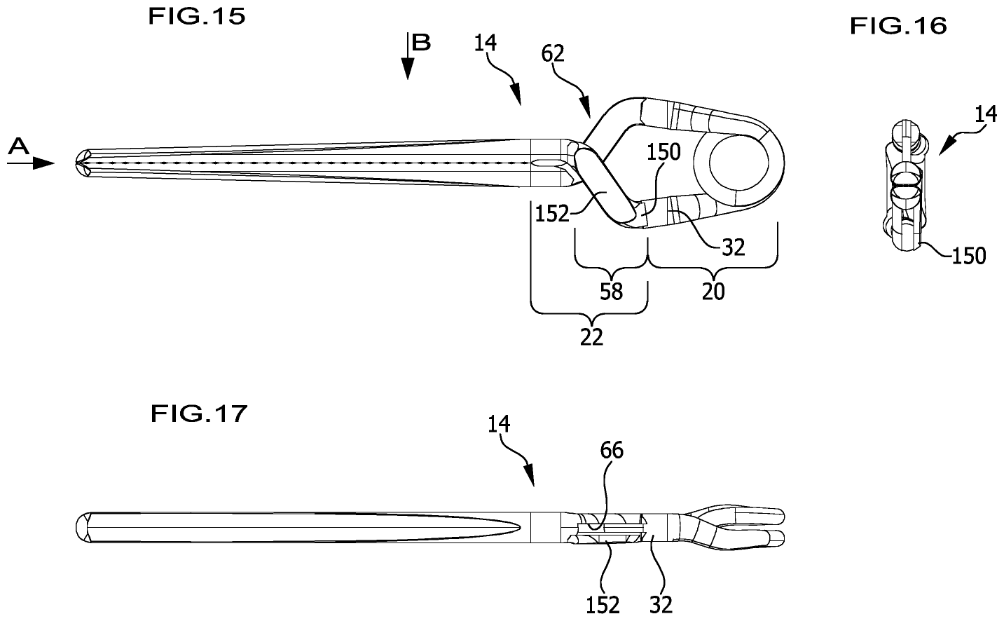

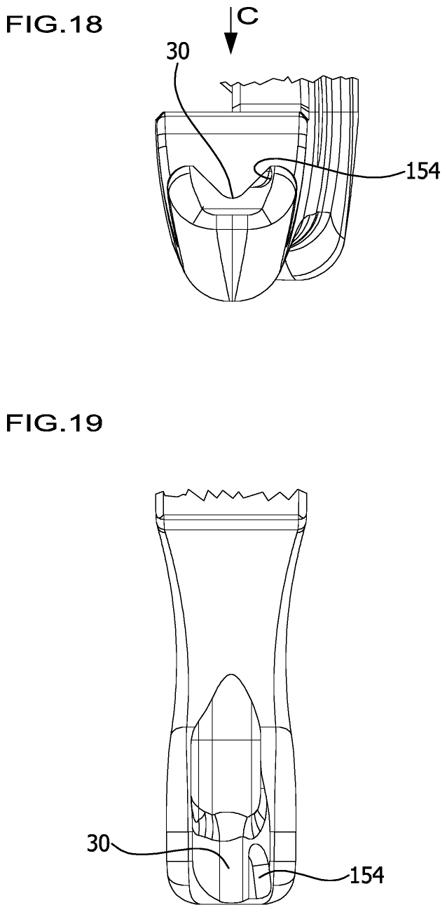

Schematically depicted in FIGS. 12 to 19 is a further embodiment of an application system 10. Except for the modifications described in more detail below, it corresponds to the embodiment of the application system 10 described above in conjunction with FIGS. 1 to 11. The reference numerals used in FIGS. 12 to 19 correspond to the reference numerals used in FIGS. 1 to 11 for corresponding parts and components.

FIGS. 12 to 14 schematically show a first application instrument 12 in cooperation with a first clip 14 of the first type corresponding thereto. The difference of the first clip 14 of the first type according to the embodiment of FIGS. 12 to 19 from the first clip 14 of the first type according to FIGS. 1 to 11 is that a laterally protruding bead 150 is formed in the transition region from the first end region 20 to the first connecting portion 22, which defines the connection region perforation 66. This bead 150 is formed, in particular, in the production of this first clip 14 of the first type, namely if the clip 14 in the region of the box lock 62 is not formed by a generative manufacturing process, but instead by soldering or welding on a lateral closure plate 152, which is inserted into a set-back portion for laterally closing the connection region perforation 66 that is formed during the reshaping of a blank from which the clip 14 is made. The bead 150 may be configured, in particular, in the form of a weld bead.

In the embodiment of the first clip 14 of the first type depicted in FIGS. 12 to 19, the bead 150 is formed on the end of the first accommodation portion 32 and projects laterally from the first accommodation portion 32 in a direction parallel to the first pivot axis 46.

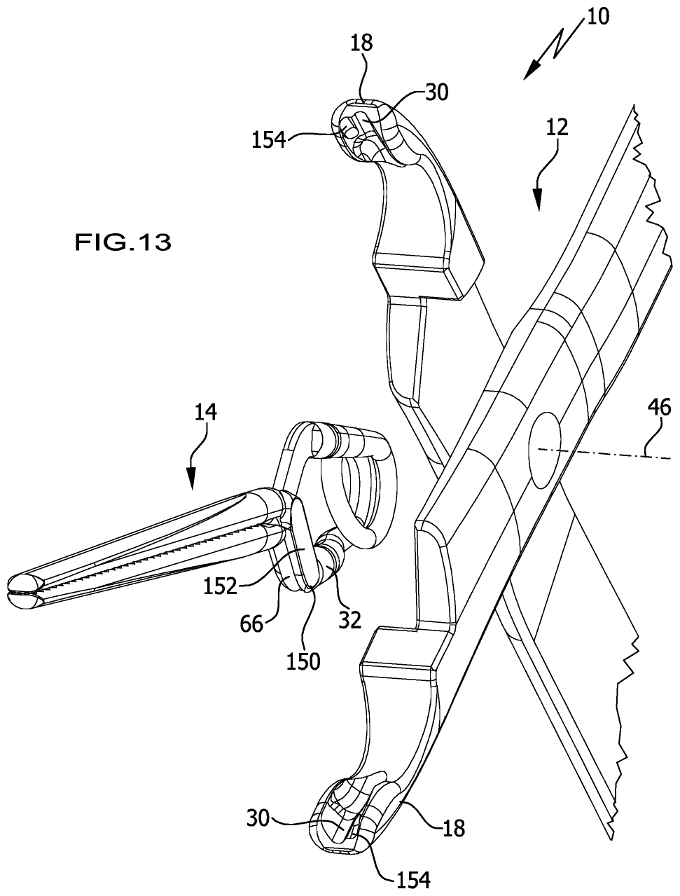

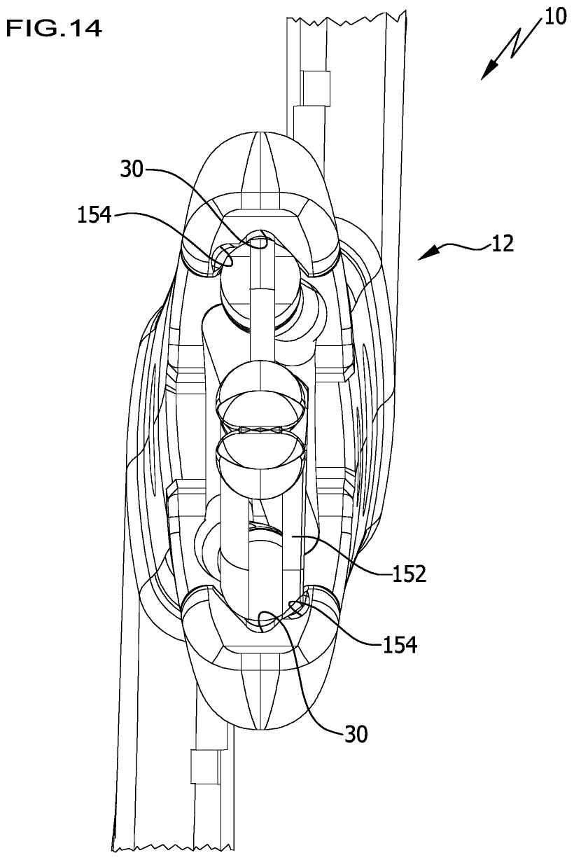

The bead 150 prevents the first clip 14 of the first type according to FIGS. 12 to 18 from being able to be completely accommodated by the first application instrument 12 according to FIGS. 1 to 11 in a defined manner and, in particular, with the accommodation portion 32 of its first connecting portion receptacles 30. An abutment of the accommodation portions 32 in the connecting portion receptacles 30, as can be clearly seen in FIG. 6, is not possible in the case of the clip 14 with the bead 150. In order to make this possible, a bead recess 154 is formed on the first application instrument 12 in accordance with the embodiment of FIGS. 12 to 19 on the first tool elements 18 of the first application instrument 12. The bead recess 154 forms a lateral deepening of the first connecting portion receptacle 30, namely on the side on which the bead 150 of the first clip 14 of the first type projects laterally. Thus, the bead recess 154 makes it possible for the first clip 14 of the first type in accordance with the embodiment of FIGS. 12 to 19 to also be able to be completely accommodated in a defined manner by the first application instrument 12 in accordance with the embodiment of FIGS. 12 to 18, as is schematically depicted, in particular, in FIGS. 12 and 14.

In an analogous manner to the first clip 14 of the first type in accordance with the embodiment of FIGS. 12 to 19, a second clip of the second type, not shown in the Figures, also has a bead. Only this bead distinguishes the second clip of the second type in accordance with the embodiment of FIGS. 12 to 19 from the second clip 16 of the second type in accordance with the embodiment of FIGS. 1 to 11.

In order for this second clip of the second type with bead to also be able to be accommodated in the connecting portion receptacles of a second application instrument, also not depicted in the Figures, bead recesses, corresponding to the bead recesses 154, for the bead on the second clip of the second type are also formed on the second end region receptacles of said second application instrument.

The bead recess 154 described in connection with the embodiment of the application system 10 in accordance with FIGS. 12 to 19 can also be provided on any application instruments for the application of medical clips, if the clips, as described, have closure plates that laterally close the connection region perforation and that are connected to the clip in the respective connecting portion by welding or soldering. Such an application instrument can, but does not have to be part of an application system 10 in order to be able to distinguish differently shaped clips from one another as described.

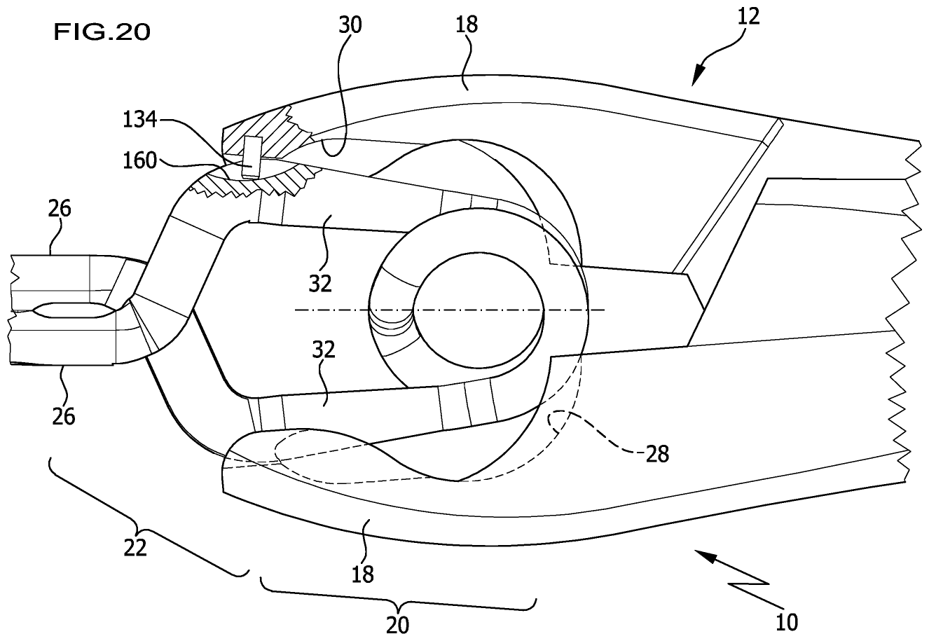

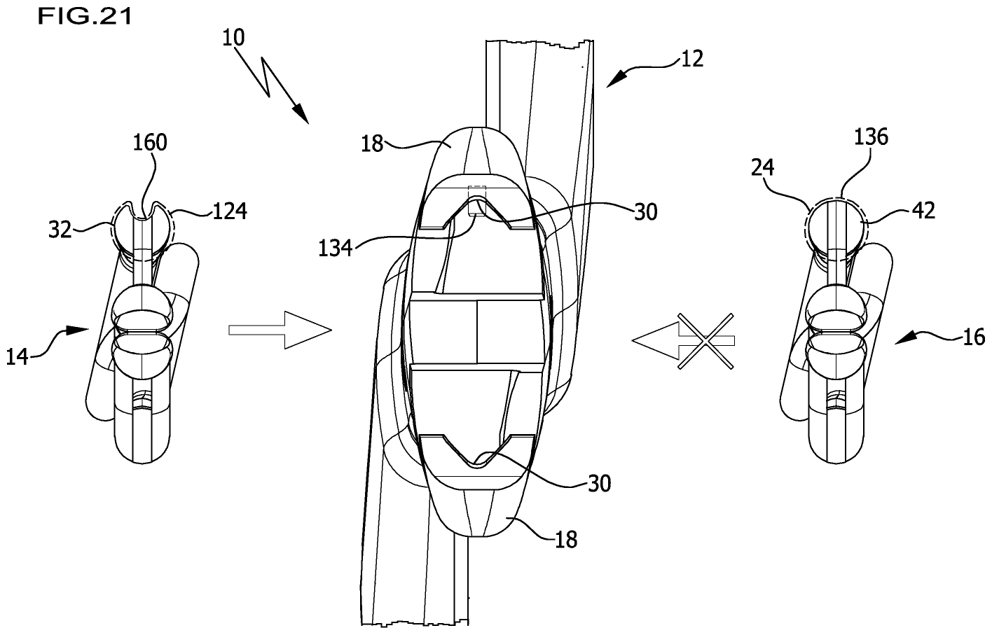

A further embodiment of an application system 10 is schematically depicted in FIGS. 20 and 21.

This application system 10 for medical clips comprises at least one first application instrument 12 for applying a first type of a medical clip 14. Only a first application instrument 12 is partially depicted in FIGS. 20 and 21.

The application system 10 further comprises at least one first medical clip 14 of the first type. In FIG. 20 and in FIG. 21 on the left, only one single first medical clip 14 is shown as an example.

The first application instrument 12 comprises two first tool elements 18 that are movable relative to one another, wherein the at least one first medical clip 14 of the first type has a first proximal end region 20 and two first connecting portions 22, which distally adjoin the first proximal end region 20 and connect the first proximal end region 20 to two clamping arms 26 of the at least one first clip 14 of the first type that are movable relative to one another. At least one of the two first connecting portions 22 defines a first volume region 124.

The two first tool elements 18 comprise between them a first end region receptacle 28 that is open in the distal direction for accommodating the first proximal end region 20 in a positive-locking or substantially positive-locking manner and each comprise a first connecting portion receptacle 30 for at least partially accommodating a respective first accommodation portion 32 of the first connecting portions 22.

The application system 12 further comprises at least one second medical clip 16 of a second type. In FIG. 21 on the right, only one single second medical clip 16 is shown as an example. The at least one second medical clip 16 comprises two connecting portions 40, which each define an accommodation portion 42.

At least one first distinguishing element 134 is arranged or formed on at least one of the two first tool elements 18 in such a way that it engages into a second volume region 136, which is defined by at least one of the two accommodation portions 42.

The first volume region 124 and the second volume region 136 overlap only partially under the constraint that the other of the two second accommodation portions is accommodated in a first connecting portion receptacle 30 of the two first tool elements 18 and the proximal end region 20 is accommodated in the first end region receptacle 28.

The clip 14 differs from the clip 16 of this embodiment in that formed on one of the two first accommodation portions 32 is a distinguishing element receptacle 160, into which the distinguishing element 134 on the first application instrument 12 engages when the proximal end region 20 is accommodated in the end region receptacle 28 and the other of the two first accommodation portions 32 is accommodated in the other connecting portion receptacle 30 of the other tool element 18.

Since no distinguishing element receptacle is formed on any of the second connecting portions 42 of the second clip 16, the distinguishing element 134, which in the embodiment depicted in the Figures is configured in the form of a protruding pin, prevents one of the second connecting portions 42 from being able to dip in the connecting portion receptacle 30 on which the distinguishing element 134 is arranged. This prevents the clip 16 from being able to be handled with the first application instrument 12. With the first application instrument 12, the two clips 14 and 16 can thus be distinguished from one another.

The embodiments described above of application systems 10 for medical clips enable a reliable differentiation of mechanically different medical clips 14 and 16 from one another in a simple manner. In particular, differently shaped end regions 20 and 38 can be used to distinguish the clips 14 and 16 from one another. Serving this purpose are the distinguishing elements 34 and 106 on the application instruments 12 and 98, which interact with the end regions 20 and 38 in such a way that in each of the application instruments 12 and 98 only one respective clip 14 or 16 can be accommodated and applied.

Confusion of the clips 14 and 16 during surgical procedures can be avoided in the described way. This is advantageous, in particular, when the clips 14 and 16 have different shapes, in particular in the region of the connecting portions 22 and 40, and define different closing forces.

Unlike, for example, in the case of color markings for associating clips with corresponding application instruments, the proposed embodiments of application systems 10 have the advantage that even under poor viewing conditions, a confusion between clips 14 and 16 on the one hand and the application instruments 12 and 98 on the other hand can be ruled out.

What is claimed is:

1. An application system for medical clips, comprising at least one first application instrument for applying a first type of a medical clip and at least one first medical clip of the first type, wherein the first application instrument comprises two first tool elements that are movable relative to one another, wherein the at least one first medical clip of the first type has a first proximal end region and two first connecting portions, which distally adjoin the first proximal end region defining a first volume region and connect the first proximal end region to two clamping arms of the at least one first clip of the first type that are movable relative to one another, wherein the two first tool elements comprise between them a first end region receptacle that is open in the distal direction for accommodating the first proximal end region in a positive-locking or substantially positive-locking manner and each comprise a first connecting portion receptacle for at least partially accommodating a respective first accommodation portion of the first connecting portions, wherein at least one first distinguishing element is arranged or formed on at least one of the two first tool elements in such a way that it engages into a second volume region, which is defined by a second proximal end region of a second medical clip of a second type, wherein two second connecting portions, each comprising a second accommodation portion, extend away from the second proximal end region in the distal direction, wherein the first volume region and the second volume region only partially overlap under the constraint that the second accommodation portions are each accommodated in a first respective connecting portion receptacle of the two first tool elements, wherein the application system comprises at least one second application instrument for applying a second type of a medical clip and at least one second medical clip of the second type, which comprises two second clamping arms that are movable relative to one another and that distally adjoin the two second connecting portions, wherein the at least one second application instrument comprises two second tool elements that are movable relative to one another, wherein the two second tool elements comprise between them an end region receptacle open in the distal direction for accommodating the second proximal end region in a positive-locking or substantially positive-locking manner and each comprise a second connecting portion receptacle for each at least partially accommodating a respective one of the second accommodation portions, wherein at least one second distinguishing element is arranged or formed on at least one of the two second tool elements in such a way that it engages into the first volume region under the constraint that the first accommodation portions are each accommodated in a respective second connecting portion receptacle of the two second tool elements.

2. The application system according to claim 1, wherein the at least one second application instrument comprises a second actuating device for moving the two second tool elements relative to one another from a second accommodation position, in which a second medical clip of the second type corresponding to the second application instrument can be accommodated with its second end region in the second end region receptacle, into a second application position, in which the two second tool elements are moved toward one another and the second clamping arms are moved away from one other for applying the second clip of the second type.

3. The application system according to claim 1, wherein the two second tool elements of the at least one second application instrument are movable relative to one another.

4. The application system according to claim 1, wherein the at least one first application instrument comprises a first actuating device for moving the two first tool elements relative to one another from a first accommodation position, in which a first medical clip of the first type corresponding to the first application instrument can be accommodated with its first end region in the first end region receptacle, into a first application position, in which the two first tool elements are moved toward one another and the first clamping arms are moved away from one other for applying the first clip of the first type.

5. The application system according to claim 1, wherein the two first tool elements of the at least one first application instrument are movable relative to one another.

6. The application system according to claim 1, wherein at least one of
a) the first accommodation portions directly or substantially directly adjoin the first proximal end region
and
b) the second accommodation portions directly or substantially directly adjoin the second proximal end region.

7. The application system according to claim 1, wherein at least one of
a) the first proximal end region comprises a first biasing element
and
b) the second proximal end region comprises a second biasing element.

8. The application system according to claim 1, wherein at least one of
a) the first clamping arms abut against one another in a first basic position of the at least one first clip of the first type and are movable away from one another against the action of the first biasing element from the first basic position into a first opening position
and
b) the second clamping arms abut against one another in a second basic position of the at least one second clip of the second type and are movable away from one another against the action of the second biasing element from the second basic position into a second opening position.

9. The application system according to claim 1, wherein at least one of
a) the first connecting portions intersect in a first connection region of the at least one first clip of the first type
and
b) the second connecting portions intersect in a second connection region of the at least one second clip of the second type.

10. The application system according to claim 1, wherein at least one of
a) at least one of the first biasing element is configured in the form of a first coil spring with at least one complete winding and the second biasing element is configured in the form of a second coil spring with at least one complete winding,
and
b) at least one of the first biasing element and the second biasing element are made by a cold forming process.

11. The application system according to claim 1, wherein at least one of
a) the at least one first medical clip of the first type defines a first clip plane, wherein the first coil spring defines a first coil spring longitudinal axis, wherein the first coil spring longitudinal axis extends perpendicularly to a first coil spring plane, and wherein the first coil spring plane is inclined relative to the first clip plane by a first angle of inclination
and
b) the at least one second medical clip of the second type defines a second clip plane, wherein the second coil spring defines a second coil spring longitudinal axis, wherein the second coil spring longitudinal axis extends perpendicularly to a second coil spring plane, and wherein the second coil spring plane is inclined relative to the second clip plane by a second angle of inclination.

12. The application system according to claim 11, wherein at least one of
a) the first coil spring is wound clockwise and wherein the first angle of inclination has a positive value
and
b) the second coil spring is wound counterclockwise and wherein the second angle of inclination has a negative value.

13. The application system according to claim 12, wherein the at least one first application instrument is configured corresponding to a first medical clip of the first type with a first coil spring wound clockwise.

14. The application system according to claim 12, wherein the at least one second application instrument is configured corresponding to a second medical clip of the second type with a second coil spring wound counter-clockwise.

15. The application system according to claim 11, wherein at least one of
a) the first angle of inclination has a value in a range between 0° and about 40°,
and
b) the second angle of inclination has a value in a range between 0° and about −40°.

16. The application system according to claim 1, wherein the at least one first distinguishing element is configured in the form of a first coding element that cooperates with any medical clip and wherein exclusively the first medical clip of the first type corresponding to the first coding element can be accommodated in the first end region receptacle of the first application instrument in a positive-locking or substantially positive-locking manner.

17. The application system according to claim 16, wherein at least one of
a) at least one of a respective one of the two first coding elements is arranged or formed on each first tool element of the first application instrument and a respective one of the two second coding elements is arranged or formed on each second tool element of the second application instrument,
and
b) at least one of the at least one first coding element is formed on the one of the two first tool elements projecting in the distal direction beyond a proximal end of the other first tool element delimiting the first end region receptacle and the at least one second coding element is formed on the one of the two second tool elements projecting in the distal direction beyond a proximal end of the other second tool element delimiting the second end region receptacle.

18. The application system according to claim 1, wherein the at least one second distinguishing element is configured in the form of a second coding element that cooperates with any medical clip and wherein exclusively the second medical clip of the second type corresponding to the second coding element can be accommodated in the second end region receptacle of the second application instrument in a positive-locking or substantially positive-locking manner.

19. The application system according to claim 1, wherein at least one of
a) the two first tool elements are convertible into one another in the region of the first end region receptacle by rotation by 180° relative to a first longitudinal axis of the first end region receptacle
and
b) the two second tool elements are convertible into one another in the region of the second end region receptacle by rotation by 180° relative to a second longitudinal axis of the second end region receptacle.

20. The application system according to claim 1, wherein at least one of the at least one first medical clip of the first type and the at least one second medical clip of the second type are configured in the form of aneurysm clips.
